(19) United States
(12) Patent Application Publication
(10) Pub. No.: US 2011/0196139 A1
(43) Pub. Date: Aug. 11, 2011

(54) PREPARATION METHOD FOR CELLULOSE PHOSPHITE COMPOUNDS

(75) Inventors: Hyun Joo Lee, Gyeonggi-Do (KR); Bang Sook Lee, Seoul (KR); Young-Woong Suh, Seoul (KR); Byoung Koun Min, Seoul (KR); Byoung Sung Ahn, Seoul (KR); Hoon Sik Kim, Seoul (KR)

(73) Assignee: Korea Institute of Science and Technology, Seoul (KR)

(21) Appl. No.: 12/909,475

(22) Filed: Oct. 21, 2010

(30) Foreign Application Priority Data
Feb. 5, 2010 (KR) 10-2010-0010954

(51) Int. Cl. C07H 1/02

(52) U.S. Cl. 536/18.6

(57) ABSTRACT

Provided is a method for preparing a cellulose phosphite compound, more particularly a method for preparing a cellulose phosphite compound whereby cellulose is phosphorylated using an ionic liquid comprising an amine-based cation and a phosphite-based anion.

Thus prepared cellulose phosphite compound has a good solubility in water and is highly valuable as concentration control agent, medicine, biomembrane, or the like.

### CROSS-REFERENCE TO RELATED APPLICATIONS

This application claims priority under 35 U.S.C. §119 to Korean Patent Application No. 10-2010-0010954, filed on Feb. 5, 2010, in the Korean Intellectual Property Office, the disclosure of which is incorporated herein by reference in its entirety.

### TECHNICAL FIELD

The following disclosure relates to a method for preparing a cellulose phosphite compound, and in particular, to a method for preparing a cellulose phosphite compound via a phosphorylation of cellulose using an ionic liquid containing an amine-based cation and a phosphite-based anion.

### BACKGROUND

Cellulose is a polymeric substance and has long been used in the form of cotton fabric, linen and paper. It is still used as fiber, membrane, film and paint and for various other industrial or household applications after chemical modification. Especially, cellulose having a phosphorus group such as cellulose phosphate or cellulose phosphite is used as a protein anchoring membrane, a heavy metal absorbent, a flame-retardant cellulose fiber, an adhesive and corrosion inhibitor. In particular, water-soluble, phosphorus-substituted cellulose may be useful as a viscosity control agent for industrial use and medicinal or cosmetic additive.

Phosphorylation of cellulose is a reaction whereby the hydroxyl group of the glucose unit of the cellulose is substituted with a phosphorus-containing group. Since cellulose is usually insoluble in water or organic solvents, only the surface group is substituted via a heterogeneous reaction or the reaction is carried out in a solvent system consisting of an organic solvent and a salt, such as NMMO, LiCl/HMPT and LiCl/DMA. Although H₃PO₄, P₂O₅, POCl₃, PCl₃, or the like is used as a phosphorylation reagent, they induce crosslinking of the polymers during the reaction and, thereby, result in decreased solubility.

U.S. Pat. No. 2,759,924 disclosed a synthesis of water-soluble cellulose using a tri-component mixture of H₃PO₄/P₂O₅/DMSO or H₃PO₄/P₂O₅/aliphatic alcohol. However, the resulting polymer has a degree of substitution (DS) smaller than 0.2 and the polymer chain tends to be broken during the reaction.

Wagenknecht et al. [Acta Polymerica, 30, 108 (1979)] performed phosphorylation of cellulose using a chlorine-based compound such as PCl₅, POCl₃ and PCl₃ and N,N-dimethylformamide (DMF) or dimethylacetamide (DMAc) as a solvent. The resulting cellulose phosphate/phosphite had a DS of about 0.3 and was partially soluble in water. However, degradation of the polymer chain and contamination by chlorine produced during the reaction was observed.

The use of cellulose acetate in preparing phosphorus-substituted cellulose has also been disclosed. For example, Wagenknecht et al. disclosed a method of manufacturing cellulose phosphate by first preparing a cellulose polymer substituted both with acetate and phosphate using cellulose acetate, tetrapolyphosphoric acid (H₆P₄O₁₃) and butylamine and then selectively removing the acetate only (Patent DE 443518, Papier 50, 712, 1996). However, considering that cellulose acetate is also prepared from cellulose, this method may require a multiple step process to manufacture cellulose phosphite.

N. Gospodinova et al. disclosed a result of cellulose phosphorylation by reacting cellulose with phosphorus acid (H₃PO₃) in urea solvent under microwave condition [Green Chemistry, 4, 220, (2002)]. This method has an advantage in that the DS can be increased to 2.89 in the reaction time of 2 to 6 h. However, it has disadvantage in that it requires a special equipment such as a microwave oven.

The inventors have made efforts to solve the problems of the existing methods. As a result, a new preparation method was developed wherein an ionic liquid including an amine-based cation and a phosphite-based anion is used as a reagent to synthesize a cellulose phosphite compound.

### SUMMARY

The present invention relates to a novel method for preparing a cellulose phosphite compound involving no degradation of the cellulose polymer chain or a side reaction such as intermolecular crosslinking.

The present invention also relates to a novel method for preparing a cellulose phosphite compound with controlled solubility in water.

In one general aspect, the present invention provides a method for preparing a cellulose phosphite compound by reacting cellulose with an ionic liquid comprising an amine-based cation and a phosphite-based anion.

In the preparation method according to the present invention, a specific ionic liquid is used as a phosphorylation reagent and also as a reaction solvent instead of a chlorine-based compound or an acid-based phosphorous compound. Accordingly, it is more environment-friendly than the conventional methods.

In the preparation method according to the present invention, the reaction is conduced in the absence of an acid, and thus degradation of the cellulose chain or a side reaction such as intermolecular crosslinking can be prevented.

The cellulose phosphite compound prepared by the method according to the present invention has 0.25 to 1.3 phosphite substitution per glucose unit of the cellulose and water solubility (at 25° C.) may be controlled in the range of 10 to 25. Thus, it is industrially valuable as water-soluble cellulose.

Other features and aspects will be apparent from the following detailed description and the claims.

### DETAILED DESCRIPTION OF EMBODIMENTS

The advantages, features and aspects of the present invention will become apparent from the following description of the embodiments with reference to the accompanying drawings, which is set forth hereinafter. The present invention may, however, be embodied in different forms and should not be construed as limited to the embodiments set forth herein. Rather, these embodiments are provided so that this disclosure will be thorough and complete, and will fully convey the scope of the present invention to those skilled in the art. The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

The present invention relates to a method for preparing a cellulose phosphite compound, comprising phosphorylating cellulose in an ionic liquid comprising an amine-based cation and a phosphite-based anion. That is, in the present invention, an ionic liquid comprising an amine-based cation and a phosphite-based anion is used as a phosphorylation reagent. Since the ionic liquid also serves as a reaction solvent, the preparation method according to the present invention brings economic gain because no additional reaction solvent is required. Moreover, whereas the existing methods involve addition of an acid compound such as hydrochloric acid or phosphoric acid to improve efficiency of phosphorylation, the preparation method according to the present invention allows easy preparation of the desired cellulose phosphite compound without pH control of the reaction solution by using a specific ionic liquid.

To describe the ionic liquid used in the present invention as a phosphorylation reagent and also as a solvent in more detail, it is an ionic compound comprising an amine-based cation represented by Chemical Formula 1 and a phosphite-based anion represented by Chemical Formula 2 and exists as liquid at room temperature:

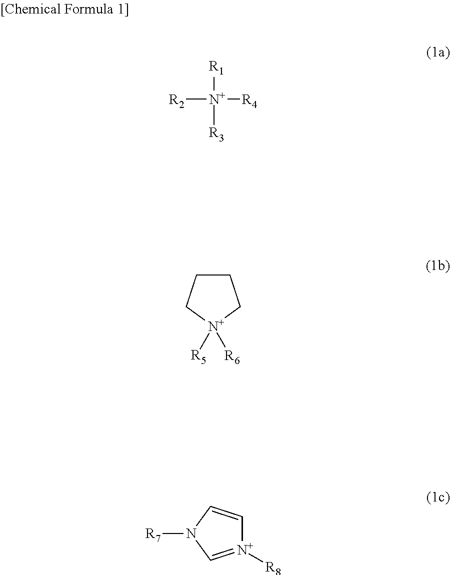

wherein R₁, R₂, R₃, R₄, R₅, R₆, R₇ and R₈, which are identical or different, represent a C₁-C₆ alkyl group, a C₁-C₆ alkylcarboxylic acid group, a C₁-C₆ alkyl ester group, a hydroxyC₁-C₆ alkyl group, dihydroxyC₁-C₆ alkyl group, a C₁-C₆ alkoxyC₁-C₆ alkyl group or a phenyl group; and

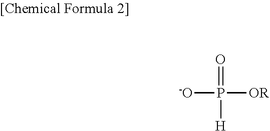

wherein R represents a hydrogen atom or a C₁-C₆ alkyl group.

In Chemical Formula 1, the substituents R₁, R₂, R₃, R₄, R₅, R₆, R₇ and R₈ of the amine-based cation may be selected from a group consisting of, for example, an alkyl group such as methyl, ethyl, n-propyl, n-butyl, n-pentyl and n-hexyl; an alkylcarboxylic acid group such as acetic acid, propionic acid and butyric acid; an alkyl ester group such as methyl acetate, ethyl acetate, ethyl propionate and ethyl butyrate; a hydroxyalkyl group such as hydroxymethyl, hydroxyethyl, hydroxypropyl, hydroxybutyl and hydroxyhexyl; a dihydroxyalkyl group such as 1,2-dihydroxyethyl; an alkoxyalkyl group such as dimethoxymethyl, methoxyethyl, ethoxyethyl, ethoxypropyl and ethoxybutyl; and a substituted or unsubstituted phenyl group. In Chemical Formula 2, the substituent R of the phosphite-based anion cation may be selected from a group consisting of: a hydrogen atom; and an alkyl group such as methyl, ethyl, n-propyl, n-butyl, n-pentyl and n-hexyl.

The ionic liquid used as the phosphorylation reagent and also as the solvent in the present invention may be synthesized easily by reacting a halide compound of the amine-based cation represented by Chemical Formula 1 with an alkali metal salt compound of the phosphite-based anion represented by Chemical Formula 2.

Hereinafter, the method for preparing a cellulose phosphite compound using the ionic liquid is described in detail.

The preparation of a cellulose phosphite compound according to the present invention is accomplished via a phosphorylation reaction whereby some of the hydroxyl group of the glucose unit of the cellulose is substituted with a phosphite anion.

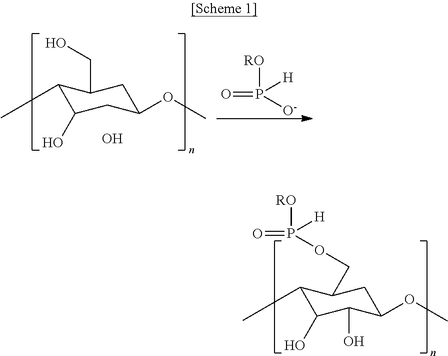

In Scheme 1, R is a hydrogen atom or a C₁-C₆ alkyl group; and n means the number of glucose units comprising cellulose. Typically, natural cellulose is a polymer compound consisting of thousands of glucose units.

The phosphorylation reaction is performed by adding cellulose to the ionic liquid and stirring while heating. The heating for the phosphorylation reaction is carried out at 100 to 200° C., preferably at 120 to 180° C. If the reaction temperature is lower than 100° C., a degree of substitution (DS) of phosphorus could be too low. Meanwhile, if it is higher than 200° C., cellulose and the ionic liquid could be degraded.

The present invention is advantageous in economy and friendliness to the environment since it requires no reaction solvent for the phosphorylation reaction. However, if necessary, an organic solvent may be used as a reaction solvent. The reaction solvent may be an organic solvent commonly used in the art. For example, a polar aprotic solvent such as dimethylsulfoxide (DMSO), N,N-dimethylformamide (DMF), N,N-dimethylacetamide (DMAc), or the like may be used.

The amount of the ionic liquid used in the phosphorylation reaction may be different depending on the DS. It is preferred to use it in an excess amount since it also serves as a solvent. Preferably, the ionic liquid is used in an amount of 1 to 20 molar equivalents per glucose unit of the cellulose. If the amount of ionic liquid is too small, homogeneous mixing becomes difficult due to increased viscosity, thereby resulting in insufficient phosphorylation. And, the use of the ionic liquid in an amount exceeding 20 molar equivalents is not favored in terms of economy.

Thus prepared cellulose phosphite compound has 0.25 to 1.3 phosphite substitution on average per glucose unit of the cellulose. Further, the solubility of the cellulose phosphite compound can be adjusted to be in the range of 10-25 g in 100 g of water at 25° C.

### EXAMPLES

The examples and experiments will now be described. The following examples and experiments are for illustrative purposes only and not intended to limit the scope of this disclosure.

### Example 1

1,3-Dimethylimidazolium methylphosphite (5 g, 300 mmol) and cellulose (0.5 g, 30 mmol based on the glucose unit) were added to a 50 mL round-bottom flask and stirred for 1 h while heating it to 140° C. Upon completion of the reaction, acetonitrile (10 mL) was slowly added. The resulting precipitate was filtered, separated and dried under vacuum to obtain white powder (0.65 g). The result showned that the prepared glucose methylphosphite had a degree of substitution (DS) of 0.67 per glucose unit of the methylphosphite (yield: 85%).

Elemental analysis: C (37.0%), H (6.0%), N (6.8%), P (8.3%).

IR (cm⁻¹): 2361 (P—H), 1211 (P═O), 1173 (P—O), 1576 (double bond in imidazolium ring), 3156 and 3109 (aromatic CH).

GPC: number average molecular weight (Mₙ)=45,793, weight average molecular weight (M_{w})=103,854, molecular weight distribution (M_{w}/Mₙ)=2.27.

In order to measure the water solubility of the prepared glucose methylphosphite, the glucose methylphosphite (0.5 g) was dissolved in distilled water (1 g) at 25° C. The dissolved portion was separated, dried and weighed to find that 0.15 g of glucose methylphosphite was dissolved. Thus, the solubility of the glucose methylphosphite prepared in Example 1 in water was 15.

### Example 2

Glucose phosphite derivatives were prepared same as in Example 1, using the ionic liquids described in Table 1. The yield of the resulting glucose phosphite derivatives, DS of the phosphite substituent per glucose unit, and solubility in water are also given in Table 1.

TABLE 1

| Ionic liquid | | | | |
|---|---|---|---|---|
| Cation | Anion | Yield (%) | DS | Solubility^{a} |
| 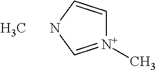 | 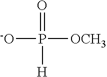 | 85 | 0.67 | 15 |
| 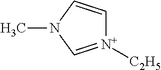 | 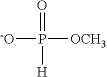 | 79 | 0.67 | 18 |
| 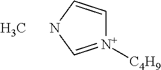 | 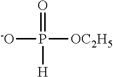 | 68 | 0.74 | 17 |
| 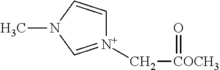 | 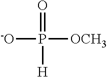 | 82 | 0.65 | 14 |
| 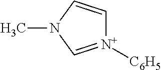 | 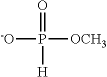 | 88 | 0.86 | 18 |
| 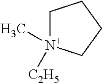 | 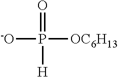 | 83 | 0.75 | 16 |
| 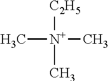 | 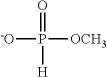 | 84 | 0.80 | 17 |

^{a}Solubility: the amount (g) of a sample dissolved in 100 g of distilled water at 25° C.

### Example 3

Glucose methylphosphite was prepared same as in Example 1, while varying reaction temperature and reaction time of the phosphorylation reaction as in Table 2. The result is also given in Table 2.

TABLE 2

| Reaction temperature | Reaction time | Yield | | |
|---|---|---|---|---|
| (° C.) | (hr) | (%) | DS | Solubility |
| 120 | 1 | 78 | 0.38 | 10 |
| | 3 | 76 | 0.55 | 13 |
| | 6 | 89 | 0.73 | 19 |
| 140 | 1 | 82 | 0.67 | 15 |
| | 3 | 84 | 0.77 | 18 |
| 160 | 1 | 88 | 0.86 | 20 |
| | 3 | 86 | 0.96 | 22 |
| 180 | 1 | 84 | 1.20 | 23 |
| | 3 | 81 | 1.53 | 25 |

### Example 4

Glucose methylphosphite was prepared same as in Example 1, using 1, 2, 10 and 20 molar equivalents of 1,3-dimethylimidazolium methylphosphite per 0.5 g of cellulose (30 mmol based on the glucose unit) in the presence or absence of a solvent. The result is given in Table 3.

TABLE 3

| | Solvent | | | | |
|---|---|---|---|---|---|
| 1,3-Dimethylimidazolium | Solvent | amount | Yield | | |
| methylphosphite (mmol) | Name | (g) | (%) | DS | Solubility |
| 600 | — | — | 92 | 0.84 | 20 |
| 300 | — | — | 54 | 0.73 | 19 |
| 60 | DMSO | 3 | 78 | 0.66 | 15 |
| 30 | DMF | 5 | 74 | 0.52 | 13 |
| 30 | NMP | 10 | 81 | 0.53 | 13 |

As demonstrated by Examples 1 to 4, the preparation method according to the present invention enables to control the DS of phosphite and water solubility of the cellulose phosphite compound.

Accordingly, the present invention can provide cellulose phosphite compounds having optimized properties for industrial use as a concentration controlling agent, medicine, biomembrane, or the like.

While the present invention has been described with respect to the specific embodiments, it will be apparent to those skilled in the art that various changes and modifications may be made without departing from the spirit and scope of the invention as defined in the following claims.

What is claimed is:

1. A method for preparing a cellulose phosphite compound, comprising phosphorylating cellulose in an ionic liquid comprising an amine-based cation and a phosphite-based anion.

2. The method for preparing a cellulose phosphite compound according to claim 1, wherein the amine-based cation of the ionic liquid is selected from the cations represented by Chemical Formula 1: 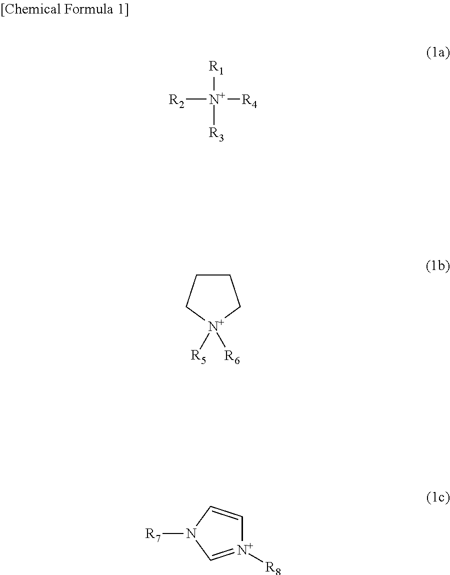
wherein R₁, R₂, R₃, R₄, R₅, R₆, R₇ and R₈, which are identical or different, represent a C₁-C₆ alkyl group, a C₁-C₆ alkylcarboxylic acid group, a C₁-C₆ alkyl ester group, a hydroxyC₁-C₆ alkyl group, dihydroxyC₁-C₆ alkyl group, a C₁-C₆ alkoxyC₁-C₆ alkyl group or a phenyl group.

3. The method for preparing a cellulose phosphite compound according to claim 1, wherein the phosphite-based anion of the ionic liquid is selected from the anion represented by Chemical Formula 2: 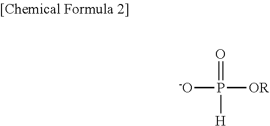
wherein R represents a hydrogen atom or a C₁-C₆ alkyl group.

4. The method for preparing a cellulose phosphite compound according to claim 1, wherein the phosphorylation is performed at 120 to 180° C.

5. The method for preparing a cellulose phosphite compound according to claim 1, wherein the phosphorylation is performed for 1 to 6 hours.

6. The method for preparing a cellulose phosphite compound according to claim 1, wherein the ionic liquid is used in an amount of 1 to 20 molar equivalents per glucose unit of the cellulose.

7. The method for preparing a cellulose phosphite compound according to claim 1, wherein the cellulose phosphite compound has a solubility (at 25° C.) in water ranging from 10 to 25.

8. The method for preparing a cellulose phosphite compound according to claim 1, wherein the cellulose phosphite compound has 0.25 to 1.3 phosphite substituted per glucose unit of the cellulose.
